(19) United States
(12) Plant Patent Application Publication
(10) Pub. No.: US 2015/0059028 P1
(43) Pub. Date: Feb. 26, 2015

(54) Rose plant named 'SPESALLE'

(50) ;

(71) Applicant: SPEK ROSE BREEDING INTERNATIONAL B.V., BOSKOOP (NL)

(72) Inventor: Erik SPEK, Boskoop (NL)

(73) Assignee: SPEK ROSE BREEDING INTERNATIONAL B.V., BOSKOOP (NL)

(21) Appl. No.: 13/987,728

(22) Filed: Aug. 26, 2013

(51) Int. Cl. A01H 5/00

(52) U.S. Cl. PLT/131

(57) ABSTRACT

A new and distinct variety of hybrid rose plant named ‘SPESALLE’ characterized by an attractive red flower with sufficient stem length, disease resistance, and acceptable productivity for the production of commercial cut flowers. ‘SPESALLE’ is characterized by a large flower with deep colored, velvety textured red petals in a very regular spiral pattern. The center petals are at the same height with the petals farther away from the center of the flower tapering down from the relatively flat time. The foliage is a dark, glossy green with a shape similar the lemon leaves.

### LATIN NAME OF THE GENUS AND SPECIES OF THE PLANT CLAIMED

Rosa L/

### VARIETY DENOMINATION

‘SPESALLE’

### BACKGROUND OF THE INVENTION

The present invention relates to a new and distinct cultivar of Rose plant, botanically known as Rosa L., hereinafter referred to by the cultivar name ‘SPESALLE’.

The new Rose ‘SPESALLE’ is a product of a breeding program conducted by the inventor, Erik Spek, in Boskoop, Netherlands. Asexual reproduction of the new hybrid rose ‘SPESALLE’ is performed by grafting or root cutting. Asexual propagation has demonstrated that the combination of characteristics as herein disclosed for the new cultivar are firmly fixed and retained through successive generations of asexual reproduction. The new cultivar reproduces true-to-type.

### BRIEF DESCRIPTION OF THE PHOTOGRAPHS

The accompanying photographs illustrate the overall appearance of the new hybrid rose ‘SPESALLE’ showing the colors as true as is reasonably possible with colored reproductions of this type. Colors in the photograph may differ slightly from the color values cited in the detailed botanical description which accurately describes the color of ‘SPESALLE’.

As shown in the photographs, ‘SPESALLE’ is characterized by a large flower with deep colored, velvety textured red petals in a very regular spiral pattern. The center petals are at the same height with the petals farther away from the center of the flower tapering down from the relatively flat time. The foliage is a dark, glossy green with a shape similar the lemon leaves.

FIG. 1 shows a partially open flower ‘SPESALLE’ on the plant.

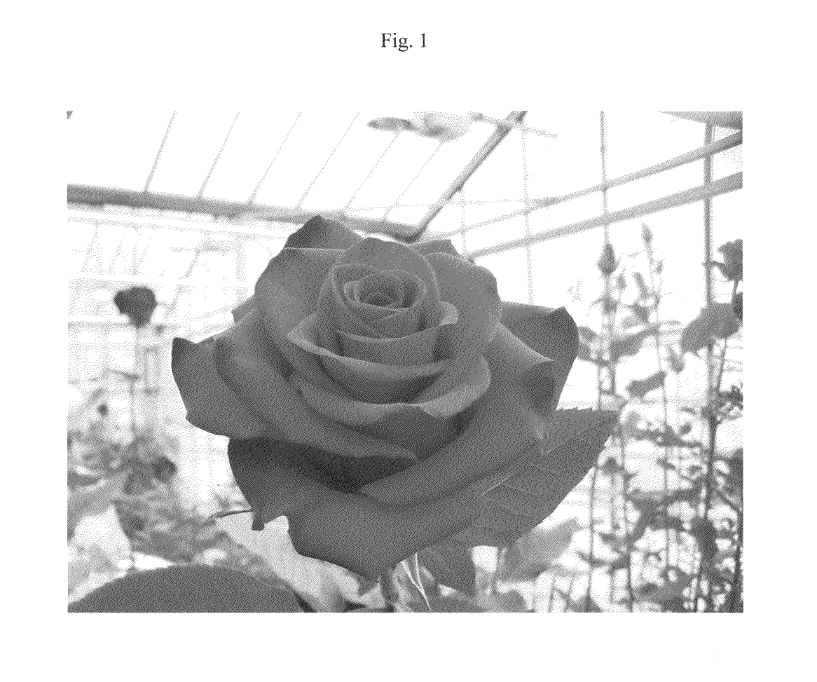

FIG. 2 shows upright growth habit of ‘SPESALLE.’

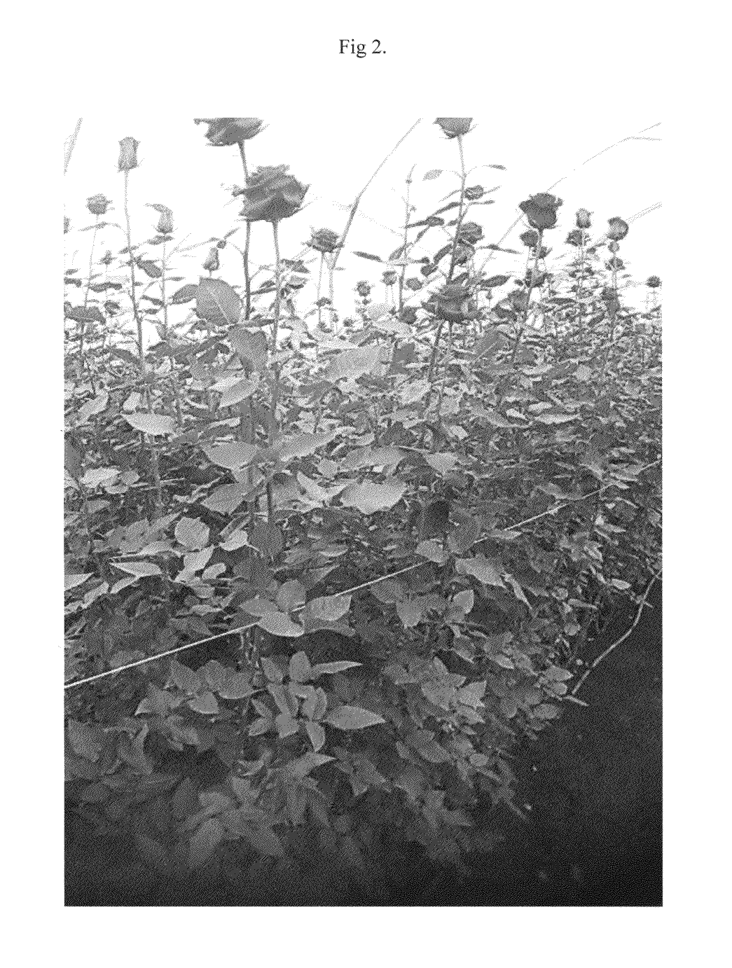

FIG. 3 shows the color and shape of young foliage growth on ‘SPESALLE’ plants in contrast with mature leaves on the ‘SPESALLE’ plants.

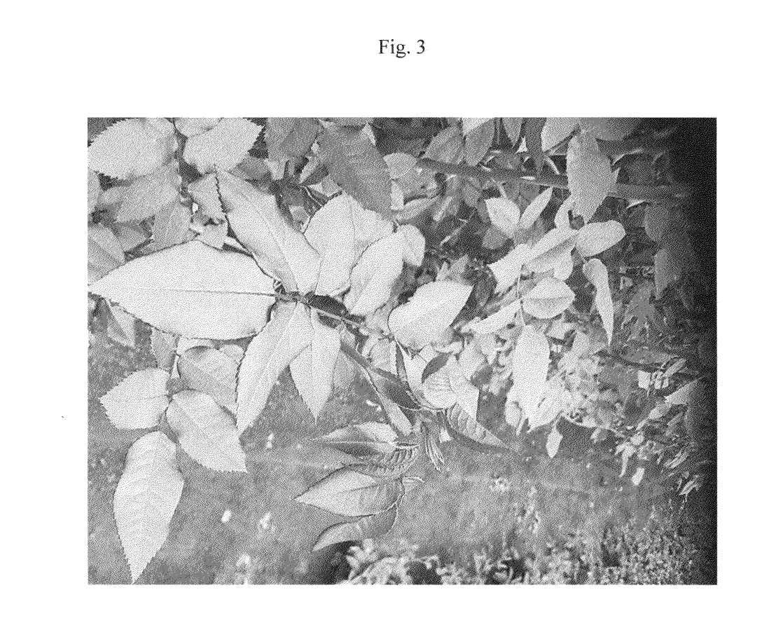

FIG. 4 shows the characteristics of the thorns and stem on the lower part of a stem on ‘SPESALLE’

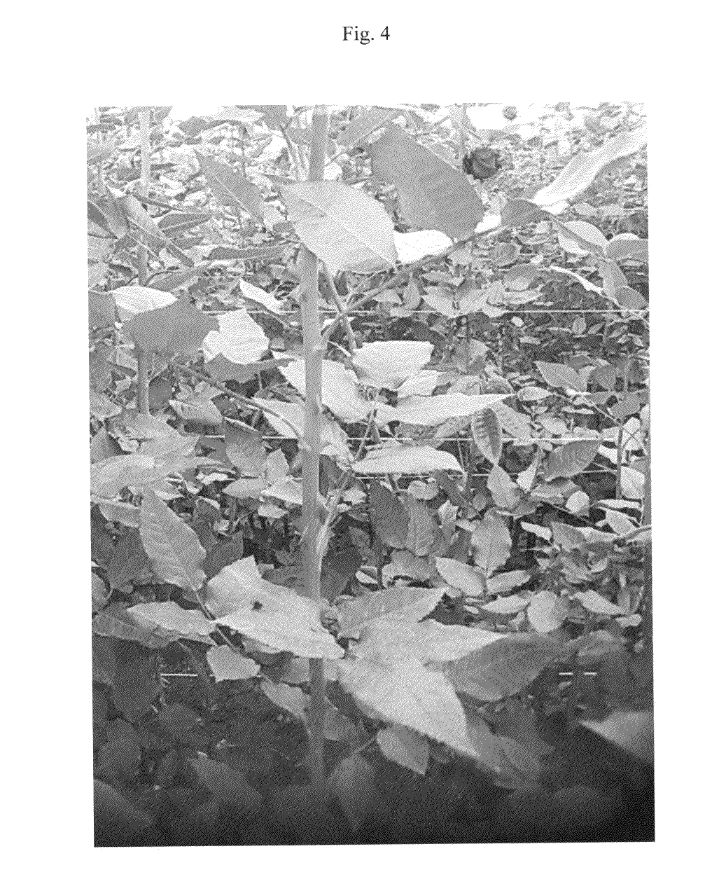

FIG. 5 shows flowers of ‘SPESALLE’ at various opening stages while still on the plants.

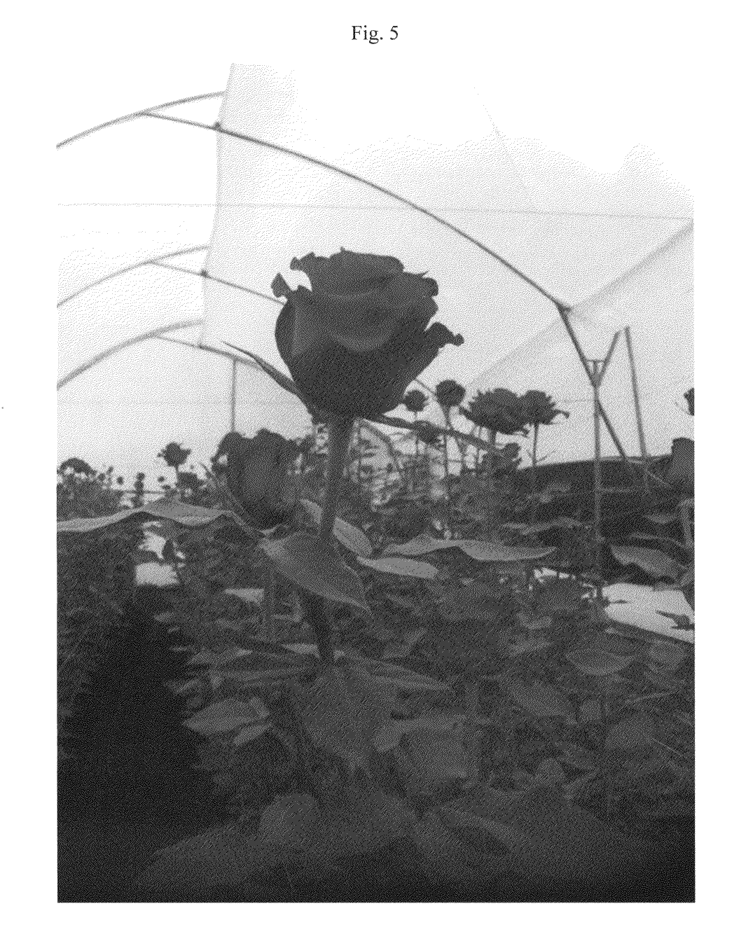

### DETAILED BOTANICAL DESCRIPTION

Plants of the cultivar ‘SPESALLE’ have not been observed under all possible environmental conditions. The phenotype may vary somewhat with variations in environment such as temperature and light intensity which can vary by season, without, however, any variance in genotype. The photographs and following observations and measurements describe plants grown near Pintag, Ecuador, under commercial practice in a polyethylene plastic covered greenhouse in a climate where the night temperatures may drop as low as 35 F but typically reach about 45 F and night and may rise to around 86 F but typically reach around 70 F day temperatures. The location is nearly 9000 feet above sea level. The observations are from plants over one year old that were propagated by patch budding on a Natal Brier rose rootstock. The representative data on the variety is for flowers produced in the high altitude tropics but shipped into the United States of America as a cut flower.
Botanical classification Rosa L.
Parentage:
   Female parent.—Proprietary Rosa sp. selection identified as code number 245 a non-patented seedling.
   Male parent.—Proprietary Rosa sp. selection identified as code number 301 a non-patented seedling.
Propagation:
   Type.—Although the cultivar can be propagated from a rooted cutting, it is recommended that it be budded on a suitable rootstock such as Rosa sp. var. Manetti or Natal Brier for greenhouse production of roses. The grafting can be as a bud, t graft stentling, or other type of graft. The rootstocks for budded plants should generally be grown for several weeks if the grafting is in the greenhouse for the plant to be actively growing to accept the bud.
   Rooting habit.—Will depend upon the rootstock used.
Plant:
   General appearance.—The cultivar ‘SPESALLE’ is a upright, freely branching grower that will produce both basal breaks and branches from the pinched plants.
   Plant height.—Typically, the plant will be managed with the flower cutting points between 30 cm and 150 cm.
   Plant diameter.—The plant will generally be managed with a diameter up to 30 cm.
   Internode length.—On stem, about 6 to 7 cm.
   Thorns.—Reddish brown color, up to about 35-40 cm from the base of the flower. The thorns under normal growing conditions are about 0.5 cm long.
   Leaves.—Generally with five leaflets, but the top leaflets may be simple and the next two or three with three leaflets. The internode length near the top is generally 4 to 5 cm. Color is generally similar to The Royal Horticultural Society Yellow Green group color 143A on the top of the leaves. The foliage is glossy. The odd pinnate leaves have ovate leaflets with a serrated edge and a rounded leaf base. Leaflets may measure up to 8 or 9 cm from base to tip.
   Cut flowers.—The cut flowers will generally have a stem length from 50 cm to 80 cm depending upon growing conditions, if the cutting is going up or down on the plant, etc. However, stem lengths of 120 cm have been measured on plants of ‘SPESALLE’.
   Flower size.—Flower bud height generally from 5.2 to 7.2 cm depending upon growing conditions and the age of the plant. Open flower diameter generally from 9 to 10 cm.
   Shape.—Inner rows of petals form comparatively flat surface area with the outer rows of petals at a progressively lower level.
   Color.—Inner petal color of open flowers is generally similar to The Royal Horticultural Society Red 41A color. The predominant color of the outer petals and top of petals of closed flowers is generally similar to The Royal Horticultural Society Red 42A color. Color may darken in the vase over time.
   Petals.—Although the exact count may vary, a typical count is about 33 to 36 petals per flower. Early flowering may have over 50 petals, but as the plant matures, the petal count is generally around 35. The edge of the petals is rounded with a slight extension to a rounded tip. If petals are not protected from UV radiation, they may experience some blackening.
   Sepals.—The sepals are narrow near the tip, approximately 4 cm long under normal conditions, and around two or three pairs of larger serrations on each side along with some very small serrations near the tip.
   Peduncles.—Peduncle is green like the rest of the stem.
Reproductive organs: None observed
Seed/fruit: None observed
Disease And pest resistance: Normal, with some susceptibility to Botrytis cinerea.

What is claimed is:

1. A new and distinct variety of rose plant named ‘SPESALLE’, as herein illustrated and described by the characteristics set forth above.
